(19) United States
(12) Patent Application Publication
(10) Pub. No.: US 2025/0277212 A1
(43) Pub. Date: Sep. 4, 2025

(54) INHIBITORS OF MIR-221 FOR THE TREATMENT OF SOLID NEOPLASMS AND PERIPHERAL NEUROTOXICITY INDUCED BY ANTICANCER DRUGS AND OTHER NEUROLOGICAL DISEASES RELATED TO MIR-221

(71) Applicant: UNIVERSITA' DEGLI STUDI MAGNA GRAECIA DI CATANZARO, Catanzaro (CZ) (IT)

(72) Inventors: Pierfrancesco TASSONE, Catanzaro (IT); Pierosandro TAGLIAFERRI, Napoli (IT); Maria Teresa DI MARTINO, Catanzaro (IT)

(73) Assignee: UNIVERSITA' DEGLI STUDI MAGNA GRAECIA DI CATANZARO, Catanzaro (CZ) (IT)

(21) Appl. No.: 18/859,323

(22) PCT Filed: May 16, 2023
(86) PCT No.: PCT/IB2023/055010

§ 371 Date: Oct. 23, 2024

(30) Foreign Application Priority Data
May 16, 2022 (IT) 102022000010130

(51) Int. Cl. C12N 15/113; A61P 35/00

(52) U.S. Cl. CPC C12N 15/113; A61P 35/00; C12N 2310/113; C12N 2310/315; C12N 2310/3231; C12N 2310/344

(57) ABSTRACT

Inhibitor of microRNA 221 (miRNA or miR-221) for the treatment of human neoplasms and/or for the treatment of peripheral neurotoxicity induced by anti-cancer drugs and/or other miR-221-related neurological distress conditions, including demyelinating diseases, and pharmaceutical composition including an inhibitor of miR-221, including LNA-i-miR-221, produced as lyophilized or in another form/variant and used with different modes of administration and dilutions, alone or in combination with other pharmacological agents.

### FIELD

The present invention relates to an inhibitor of miR-221, preferably but not exclusively LNA-i-miR-221, for the treatment of human neoplasms and peripheral neurotoxicity induced by previous anticancer treatments and other neurological distress conditions related to miR-221.

### BACKGROUND

Solid tumors have a heavy human and social impact and, at the same time, a significant financial burden on all health systems. Despite important advances in the treatment of some cancers, for many others the goal of treatment still represents an “unmet need”. Therefore, new drugs and/or new anticancer strategies are highly desirable.

There are numerous limitations to the optimal management of advanced solid tumors. Molecular targeted therapies (MTTs), for example, induce substantial benefits only in some cancers, at the cost of new and severe toxicities and, even in these cases, the disease, after a period of response, often progresses towards a state of refractoriness. In this context, microRNA-based therapies (miRNAs) may be a worthy option for extensive clinical experimental exploration with a view to overcoming some limitations of available anticancer therapies. The use of inhibitors of miRNA may represent an important option, for example, in refractory tumors that do not express “driver” molecular lesions capable of driving the evolution of the disease and that are not attackable with targeted treatments, but that have oncogenic, overexpressed and hyper functional miRNAs, which play a crucial role in the development and progression of the disease. In such pathological conditions, the use of an inhibitor of miRNA, alone or in combination with other miRNA mimics or inhibitors of miRNA, or with other anticancer agents may represent a therapeutic strategy worthy of attention and exploration.

The key role of miR-221 in disease development and progression mechanisms has been demonstrated in numerous hematologic and solid tumors with extensive preclinical testing by the Applicant and by other international research groups.

The use of inhibitors of miR-221 has been described for example by Park et all. (Park J. K. et al. Pancreas. 2009) which demonstrated that the use of an antisense oligonucleotide capable of selective targeting of miR-221 is capable of inhibiting the development of hepatocarcinoma. In addition, Callegari et all. demonstrated that treatment with an anti-miR-221 oligonucleotide in mouse models of hepatocellular carcinoma reduces the number and size of liver tumor nodules. In vitro, in pancreatic cancer cells, the use of an inhibitor of miR-221 has been shown to suppress its proliferative capacity. In addition, numerous studies support the use of inhibitors of miR-221/222 for a “rescue” of a chemo-sensitive state. Despite all this information currently available, no clinical evidence on the use of inhibitors of miR-221 has yet been published either in abstract form or in full publication or in patent filing form.

The Applicant has developed an innovative inhibitor of miR-221, called LNA-i-miR-221, described in the patent applications U.S. Pat. No. 9,404,111, EP13705584.4 and the Italian patent IT0001429326. The patent applications and patents cited concern the antisense oligonucleotide which represents a “First-in-Class agent” initially developed for the experimental treatment of multiple myeloma.

From an analysis of all available literature, including also known patent documents, there are currently no specific clinical-therapeutic applications with the use of inhibitors of miR-221 of any chemical nature in patients with solid or hematological neoplasms. Consequently, there are currently no data referring to clinical use also of the inhibitor of miR-221, called LNA-i-miR-221, proposed by the Applicant, in the treatment of solid neoplasms and in particular in colorectal cancers. Furthermore, there is currently no clinical-experimental evidence relating to the use of inhibitors of miR-221 of any chemical nature and, in this case, also of the construct proposed by the Applicant, LNA-i-miR-221, for the treatment of peripheral neurotoxicity induced by previous anticancer treatments of different nature or for the treatment of other pathological conditions of a neurological nature to be related to miR-221.

### SUMMARY

Purpose of the present invention is, on the basis of the first clinical experimental evidence obtained by the Applicant, to support the use of an inhibitor of miR-221 used in humans in cancer patients, preferably the “first-in-class” LNA-i-miR-221, for the treatment of human neoplasms, including colorectal neoplasms, as a single agent also potentially usable in combination with other drugs.

It is also a purpose of the present invention to support the use of inhibitors of miR-221, in particular LNA-i-miR-221, on the basis of the first clinical experimental evidence obtained by the Applicant, for the treatment of peripheral neurotoxicity induced by previous anticancer treatments of various nature (for example: platinum derivatives, taxanes, vinca alkaloids and others) and for the treatment of other pathological conditions of a neurological nature linked to miR-221, including demyelinating diseases.

It is a further purpose of the present invention to provide a specific pharmaceutical preparation comprising LNA-i-miR-221 for the treatment of human neoplasms and neurological distress conditions related to miR-221.

According to the present invention an anti-miR-221 (LNA-i-miR-221) clinical grade pharmaceutical preparation is provided and also a clinical use of such an agent for the treatment of solid human neoplasms for the induction of antitumor effects and for the treatment of peripheral neurotoxicity induced by previous antiblastic treatments.

### BRIEF DESCRIPTION OF THE FIGURES

For a better understanding of the present invention, a preferred embodiment is now described, by way of non-limiting example only, with reference to the figures, in which:

FIG. 1 illustrates and graphically summarizes the responses obtained from a clinical trial of the pharmaceutical preparation LNA-i-miR-221 as part of a “First-in-Human” phase I clinical trial containing the inhibitor according to the invention;

FIG. 2 shows the radiological outcome of LNA-i-miR-221 treatment in a patient who, after obtaining a therapeutic response, was further treated with other subsequent cycles, based on the provisions of the clinical protocol. The figure demonstrates quantifiable tumor regression according to RECIST criteria as partial response (PR). The patient has a refractory colorectal neoplasm, previously undergoing several lines of therapy with conventional chemotherapy regimens;

FIGS. 3A-3D show the antitumor activity induced by treatment with LNA-i-miR-221 in in vitro colon cancer experimental models, based on experimental data obtained a reduction in miR-221 levels is highlighted (FIG. 3A) and a reduction in colon cancer cell proliferation (FIG. 3B) and an increase in the expression levels of target genes both at the transcriptional (FIG. 3C) and protein level (FIG. 3D);

FIGS. 4A-4C show the results of treatment with 25 mg/kg of LNA-i-miR-221 in xenograft mouse models (colon cancer cell lines implanted in immunocompromised mice), a significant regression of tumor growth is highlighted (FIG. 4A), as well as a significant reduction of miR-221 levels (FIG. 4B) and an increase of its targets (FIG. 4C) in the explanted tumors.

The sequences and state of the art of miR-221 are available under the ID hsa-miR-221 (www.microrna.org).

### DETAILED DESCRIPTION

Object of the invention is a new medical use of a miR-221 inhibitor, preferably but not limited to LNA-i-miR-221, a chemically modified single-stranded oligonucleotide, capable of specifically binding and inhibiting mature miR-221, i.e. the endogenous 22 nucleotide molecule.

More specifically, an inhibitor of microRNA 221 (miRNA or miR-221) for the treatment of human neoplasms and/or for the treatment of peripheral neurotoxicity induced by anti-cancer drugs and/or other conditions of neurological distress conditions related miR-221, including to demyelinating disease conditions, is an object of the present invention.

Further subject of the present invention is a pharmaceutical composition comprising an inhibitor of miR-221, including but not limited to LNA-i-miR-221, produced as a lyophilized or in another form/variant and used with different modes of administration and dilutions, alone or in combination with other pharmacological agents.

The inhibitor of microRNA 221 (miR-221) has given promising results in the trials carried out by the Applicant, both in terms of effect on solid tumors and of the simultaneous onset of neurotoxic effects linked to the use of anticancer drugs used on the same patient or to conditions of neurological suffering related to miR-221.

LNA-i-miR-221 is a Locked Nucleic Acid (LNA), a 13 nucleotide long antisense oligonucleotide, with the sequence:

(SEQ ID No 1)
5′-CAGACAATGTAGC-3′.

Such oligonucleotide is synthesized with the fully phosphorothioate sugar/phosphate scaffold, and is complementary to nucleotides 66-78 of the miR-221 stem loop, the secondary structure that miR-221 assumes in the cell. The Applicant has demonstrated that such an antagonist effectively inhibits the activity of endogenous miR-221 in several solid tumors, and in particular in colorectal tumors.

For this purpose, the Applicant carried out a first-in-human, open-label study at increasing doses, on 17 patients with refractory advanced solid oncological disease and in radiological progression after one or more lines of therapy, with a limited life expectancy, acceptable “performance status”, marrow, liver and kidney function.

The results of the trial did not allow to identify the maximum tolerated dose (MTD) of LNA-i-miR-221 but allowed to establish the recommended dose for phase II studies and to determine an excellent safety profile in the absence of Grade 3 and 4 LNA-i-miR-221 toxicity events to be attributed to the drug in question.

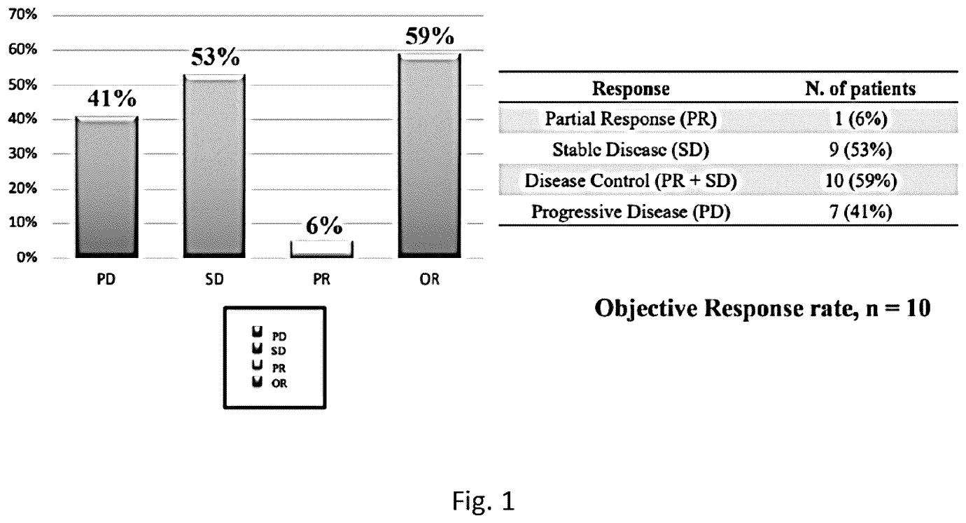

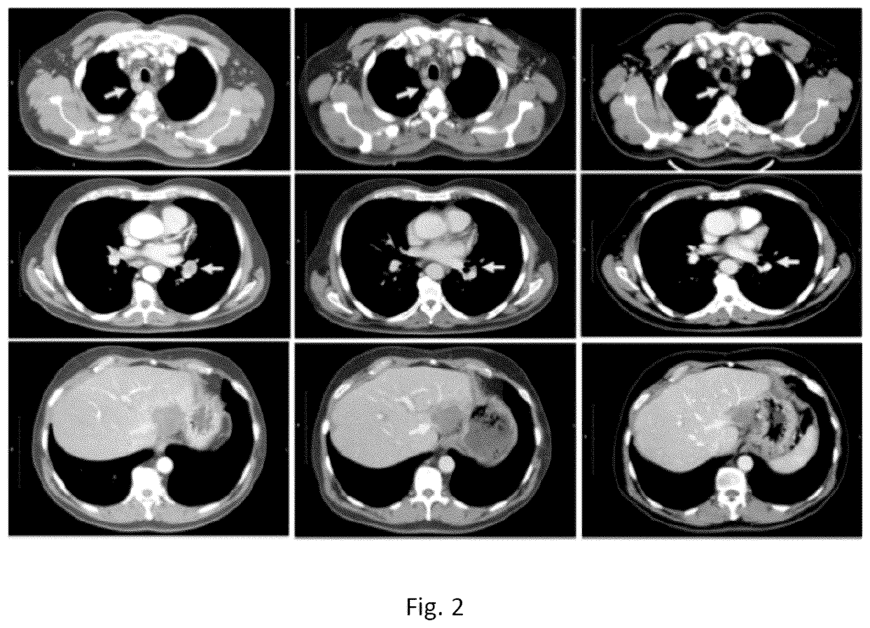

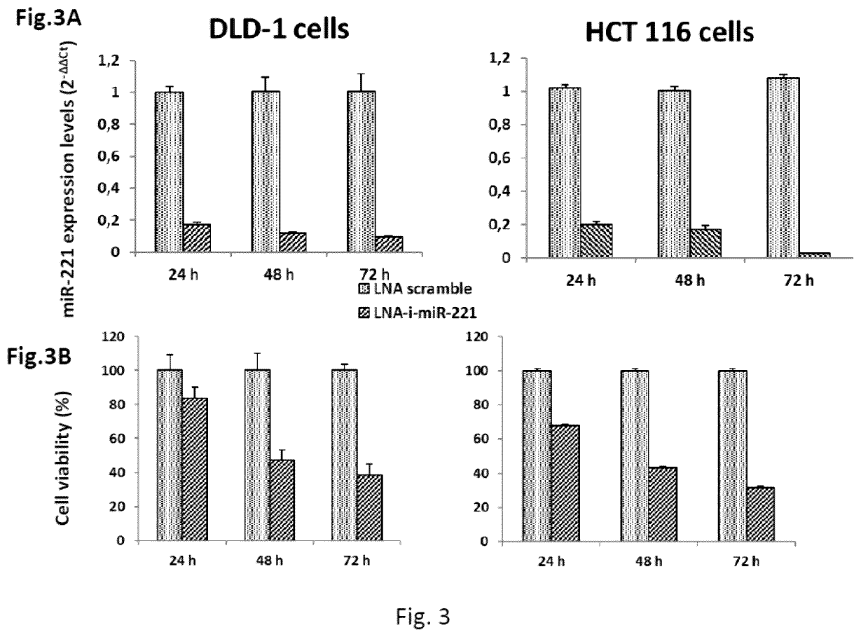

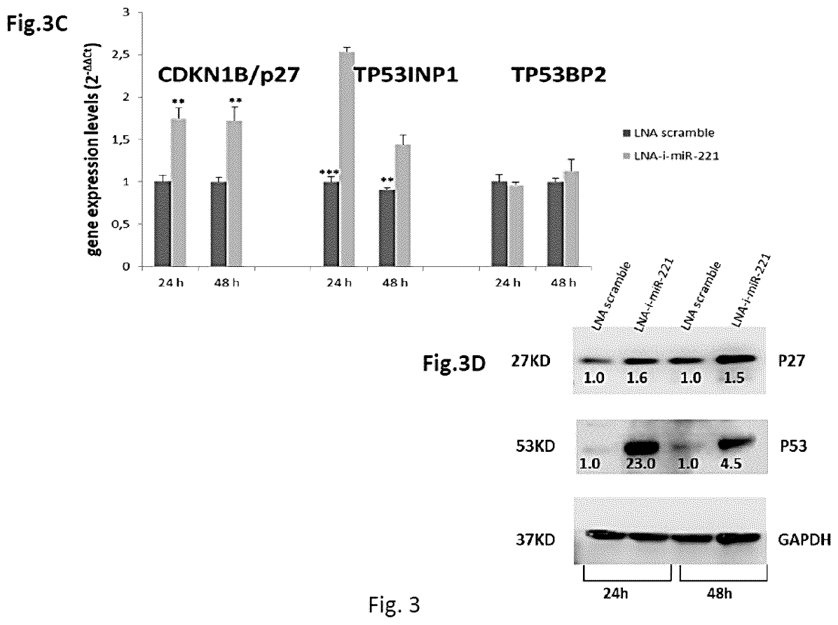

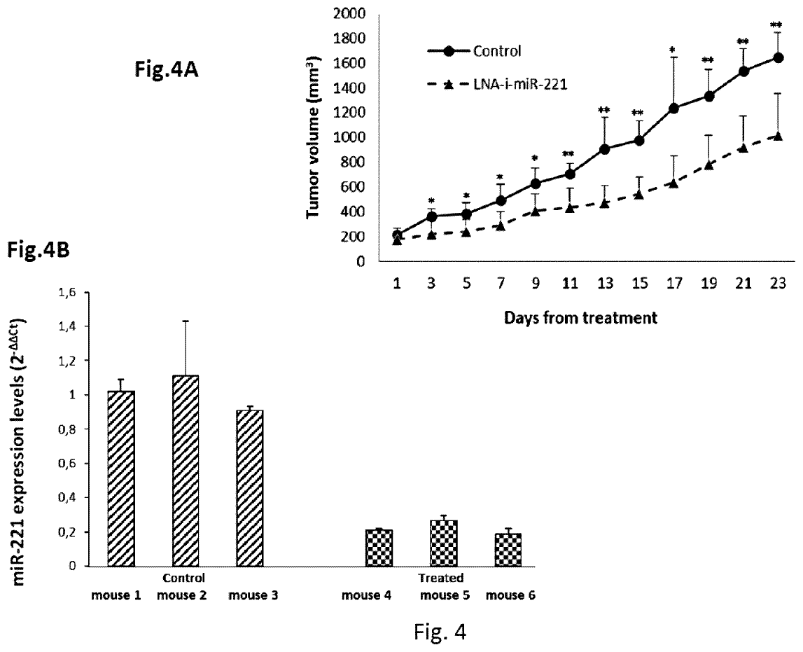

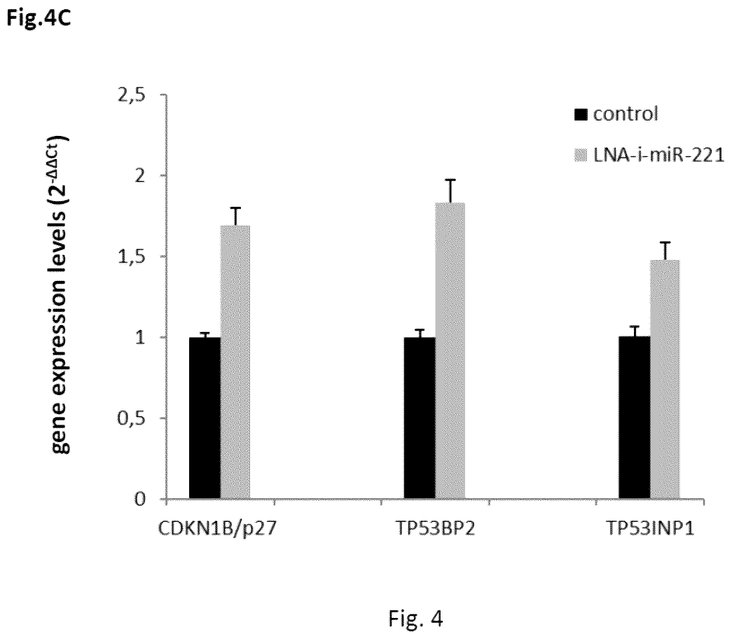

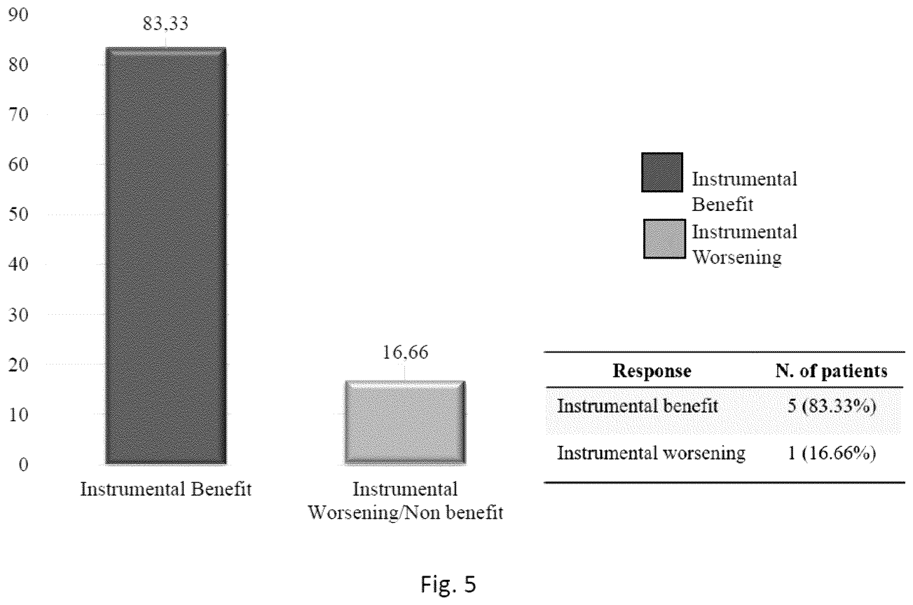

Some of the experimental results are shown in FIGS. 1-2. In the experiment to which the reported results refer, the miR-221 inhibitor used, called LNA-i-miR-221, has more specifically formula +C*A*G*+A*+C*A*+A* T*+G*T*+A*+G*C where symbol “+” indicates the position of the locked nucleotide (LNA) and symbol “*” indicates the phosphorothioate bond.

The detection of LNA-i-miR-221 in the blood and urine samples of a patient, was carried out using a validated mass spectrometry method for humans, according to protocols previously set up for the animals, suitable for pharmacokinetic assessments. For each enrolled patient, blood samples were taken at different timing on days 1 to 6 of the treatment cycle. Urine samples were collected before the dose and at different times on the first day of the treatment cycle and up to 6 days after the first treatment.

The results of the pharmacokinetic evaluation in the dose escalation study, with dose increase from 0.5 to 5 mg/kg in 5 cohorts, show that LNA-i-miR-221 is rapidly eliminated from the plasma compartment and distributed in tissues at all dose levels. The highest blood concentration was observed, as expected after intravenous administration, at the end of the infusion, with some exceptions in which Tmax was observed 15 minutes later, probably due to analytical variability as, in these cases, the concentrations were close to those observed at the end of the infusion. For the four administrations in all cohorts, mean plasma terminal half-life values of LNA-i-miR-221 ranged from 1.26 to 5.43 hours. This is probably not the true terminal half-life but more likely the half-life of the initial mixed distribution/elimination phase, as also suggested by the results of the analysis of LNA-i-miR-221 in urine. No significant differences were observed during the treatment period in terms of plasma exposure parameters (comparing respectively Cmax and AUCₜₗₐₛₜ in the four administrations, tab.1). This also suggests that no obvious changes in systemic clearance occurred during the repeated treatment interval. In conclusion, the results obtained in plasma indicate a non-linear pharmacokinetics in the dose range explored in this clinical study. In fact, an increase more than proportional to the dose of plasma exposure to LNA-i-miR-221 was observed, based on Cmax/dose, AUCtlast/dose and total clearance from 0.5 to 5 mg/kg, indicating adequate bioavailability to obtain therapeutic effects.

TABLE 1
Mean plasma pharmacokinetic parameters at each treatment dose

| | | | | HL | | | | |
|---|---|---|---|---|---|---|---|---|
| Dose | | Tₘₐₓ | Cₘₐₓ | Lambda z | AUCₗₐₛₜ | AUCₜₗₐₛₜ/Dose | CI | Vz |
| (mg/kg) | cohort | (hours) | (ng/mL) | (hours) | (h*ng/ml) | (mg/ml)/(mg/kg) | (ml/h/kg) | (ml/kg) |
| 0.5 | 1 | 0 | 1966.6 | 1 | 1752 | 3451 | 259 | 350.86 |
| 1 | 2 | 0 | 2693.2 | 2 | 3004 | 3125 | 298 | 670.64 |
| 2 | 3 | 0 | 5774.7 | 1 | 6080 | 3040 | 286 | 574.86 |
| 3 | 4 | 0 | 10840.3 | 2 | 12331 | 4110 | 121 | 500.14 |
| 5 | 5 | 0 | 20979.2 | 5 | 33034.0 | 6607 | 153 | 1068.57 |

In the analysis of the radiological responses obtained according to RECIST criteria, the first cohort was excluded since the patients had been treated with two successive cycles and the imaging was not comparable with the imaging of the subsequent cohorts. After the first court of 3 patients, in fact, the protocol was amended by reducing from two to a single cycle to facilitate the participation of patients in the study. The analysis of responses was therefore conducted only on cohorts II, III, IV and V. The results obtained in the 4 cohorts show signs of anticancer activity with stable disease (SD) without evidence of progression in 9 patients (52.9%) and partial response (PR) in 1 (5.9%) patient with colorectal cancer (DS+PR total: 58.8% patients with evidence of clinical benefit) (FIG. 1).

Furthermore, during the trial conducted by the Applicant and aimed at evaluating the safety and anti-tumor activity of LNA-i-miR-221, an obvious clinical benefit was found, as an absolutely unexpected event, on symptoms referable in the first instance to late neurotoxicity induced by previous treatments with anti-tumor drugs, which materialized in a partial or total restoration of peripheral sensitivity and fine motor skills.

On a subset of patients, it was possible to perform specific procedures for the instrumental deepening of this phenomenon. To evaluate nerve conduction along the afferent somatic-sensory pathways, the study of somatic-sensory evoked potentials (SSEP) and electromyography (EMG) was carried out.

Data analysis was performed after completion of enrollment procedures, excluding some patients with brain metastases and other patients who had no symptoms of neurotoxicity at the time of enrollment. Overall, 6 patients were considered eligible for evaluation. Of these, 5 out of 6 patients obtained a clinical and instrumental benefit with significant improvement in nerve conduction demonstrated by instrumental evaluations (SSEP and EMG) (FIG. 5). In addition, among cases reporting clinical/instrumental benefits from LNA-i-miR-221 treatment, a patient was instrumentally characterized as having a baseline condition of sensorimotor demyelinating neuropathy. Precisely this patient reported the most significant improvement compared to his baseline condition when compared to the other 4 patients belonging to the same subgroup that benefited specifically on the peripheral sensory/motor system.

FIG. 5 illustrates and graphically summarizes the neurological analysis responses obtained from a clinical trial of the pharmaceutical preparation LNA-i-miR-221 in the context of a “First-in-Human” phase I clinical trial, containing the inhibitor according to the invention. FIG. 5 illustrates that one patient of six (16.6%) patients evaluated by instrumental analysis (SSEP and EMG) has worse nerve conduction, while 5 patients (83.33%) show improvement in nerve signal conduction.

The present invention therefore also relates to the medical use of LNA-i-miR-221 in the treatment of late peripheral neurotoxicity from previous treatments with conventional anticancer drugs, including platinum derivatives (cisplatin, carboplatin and oxaliplatin) and taxanes, and other pathological conditions of a neurological nature linked to miR-221, including demyelinating disease.

The present invention relates to a pharmaceutical formulation comprising LNA-i-miR-221 without excipients and produced as a lyophilized, and dosed with an amount of 35 mg of active ingredient per vial, wherein the addition of 2 ml of saline (0.9% NaCl) to the lyophilized gives a solution of 17.5 mg/ml.

According to one aspect of the invention, in the pharmaceutical composition the miR-221 inhibitor is LNA-i-miR-221 produced as lyophilized and diluted in saline in the range of 10 mg/l to 25 mg/l, preferably 17.5 mg/ml.

According to one aspect of the invention, no excipients have been added to the pharmaceutical formulation.

According to one aspect of the invention, the pharmaceutical composition is subjected to bolus infusion without filtration through a controlled infusion pump in a final volume of 100 ml for 30 min.

According to one aspect of the invention, the pharmaceutical composition according to the invention is administered by intravenous bolus.

According to another aspect of the invention, the pharmaceutical composition according to the invention is administered subcutaneously or aerosol.

The pharmaceutical composition is preferably reconstituted at a concentration of 17.5 mg/ml using sterile saline (0.9% NaCl) for injectable preparations.

According to the present invention, an inhibitor of microRNA 221 (miRNA or miR-221) is developed and claimed for the clinical treatment of patients with human malignancies refractory to anticancer treatments and/or for the treatment of patients suffering from peripheral neurotoxicity induced by previous anticancer treatments and/or other neurological suffering conditions related to miR-221 dysregulation, including demyelinating disease conditions.

According to one aspect of the invention, an inhibitor of microRNA miR-221, called LNA-i-miR-221, having formula +C*A*G*+A*+C*A*+A*T*+G*T*+A*+G*C wherein the symbol “ +” indicates the position of the locked nucleotide (LNA) and the symbol “*” indicates the phosphorothioate bond, is claimed for the treatment of patients carrying human neoplasms refractory to previous therapies, including colorectal tumors, and/or for the treatment of patients suffering from peripheral neurotoxicity induced by previous anticancer treatments and/or other neurological suffering conditions linked to miR-221 dysregulation, including demyelinating disease.

According to one aspect of the invention, the use of an inhibitor of miR-221 microRNA, said LNA-i-miR-221, with formula +C*A*G*+A*+C*A*+A*T*+G*T*+A*+G*C wherein the symbol “+” indicates the position of the locked nucleotide (LNA) and the symbol “*” indicates the phosphorothioate bond, is claimed in the treatment of patients carrying refractory colorectal neoplasms to anticancer treatments.

According to one aspect of the invention, the use of an inhibitor of miR-221 microRNA, said LNA-i-miR-221, with formula +C*A*G*+A*+C*A*+A*T*+G*T*+A*+G*C wherein the symbol “+” indicates the position of the locked nucleotide (LNA) and the symbol “*” indicates the phosphorothioate bond, is claimed in the treatment of patients suffering from peripheral neurotoxicity induced by previous anticancer treatments and/or other neurological suffering conditions linked to dysregulation of miR-221, including demyelinating disease pictures.

According to one aspect of the invention, the pharmaceutical composition comprises an inhibitor of miR-221, including LNA-i-miR-221, produced as a lyophilized or in another form/variant and used with different modes of administration and dilutions, alone or in combination with other pharmacological agents.

According to one aspect of the invention, the pharmaceutical composition comprises an inhibitor of miR-221 produced as lyophilized and diluted in saline.

According to one aspect of the invention, the pharmaceutical composition comprises an inhibitor of miR-221 which is LNA-i-miR-221, produced as a lyophilized and diluted in saline in the range of 10 mg/l to 25 mg/l.

According to one aspect of the invention, a pharmaceutical composition is developed for use in the treatment of colorectal cancers refractory to previous anticancer therapies and/or for use in the treatment of patients suffering from peripheral neurotoxicity induced by previous anticancer treatments and/or other conditions of neurological distress conditions related to dysregulation of miR-221, including demyelinating disease pictures, comprising the inhibitor LNA-i-miR-221 produced as lyophilized and diluted in saline at 17.5 mg/l, administered intravenously.

According to one aspect of the invention, the pharmaceutical composition, and possible variants thereof, comprises a miR-221 inhibitor, including LNA-i-miR-221, in the presence of excipients of different nature or conveyed with vectors of different nature for use in the treatment of patients bearing human neoplasms refractory to previous anticancer treatments, including colorectal neoplasms, alone or in combination with other drugs administered intravenously or by other routes.

According to one aspect of the invention, the pharmaceutical composition comprises an inhibitor of miR-221, including LNA-i-miR-221, in the presence of excipients of different nature or conveyed with vectors of different nature for use in the treatment of patients suffering from peripheral neurotoxicity induced by previous anticancer treatments and/or for the treatment of other neuropathic disorders attributable dysregulation of miR-221, including demyelinating diseases, alone or in combination with other drugs administered intravenously or by other method of administration.

According to one aspect of the invention, the pharmaceutical composition for the treatment of patients having refractory solid neoplasms, and adapted to be administered intravenously for 4 consecutive days.

Finally, it is clear that the action of the LNA-i-miR-221 inhibitor in anti-cancer terms together with the benefit for the treatment of peripheral neurotoxicity, can benefit from potential combinations with other drugs, with potential additive/synergistic effects, therefore, modifications or variants of the treatment modalities also with other formulations have to be within the protective scope of the present invention, as defined in the appended claims.

1-10. (canceled)

11. An inhibitor of microRNA 221 (miRNA or miR-221) for the clinical treatment of patients with human neoplasms refractory to previous therapies, including colorectal cancers, and/or for the treatment of patients with peripheral neurotoxicity induced by previous anticancer treatments and/or other neurological distress conditions related to dysregulation of miR-221, including demyelinating disease, said inhibitor of microRNA miR-221, called LNA-i-miR-221, having formula 5′-CAGACAATGTAGC-3′ (SEQ ID No 1).

12. Composition containing an inhibitor of microRNA miR-221, called LNA-i-miR-221, having formula +C*A*G*+A*+C*A*+A*T*+G*T*+A*+G*C wherein the symbol “+” indicates the position of the locked nucleotide (LNA) and the symbol “*” indicates the phosphorothioate bond, for the treatment of patients with human colorectal neoplasms refractory to anticancer therapies.

13. Composition containing an inhibitor of microRNA miR-221, called LNA-i-miR-221, having formula +C*A*G*+A*+C*A*+A*T*+G*T*+A*+G*C wherein the symbol “+” indicates the position of the locked nucleotide (LNA) and the symbol “*” indicates the phosphorothioate bond, for the treatment of patients with peripheral neurotoxicity induced by previous anticancer treatments and/or other neurological distress conditions related to dysregulation of miR-221, including demyelinating disease.

14. A pharmaceutical composition comprising an inhibitor of miR-221, including LNA-i-miR-221, according to claim 11, said inhibitor being produced as a lyophilized or in another form/variant and used with different methods of administration and dilution, alone or in combination with other pharmacological agents.

15. The pharmaceutical composition according to claim 14 wherein said inhibitor of miR-221 is produced as a lyophilized and diluted in saline.

16. The pharmaceutical composition according to claim 14 wherein said inhibitor of miR-221 is LNA-i-miR-221 produced as a lyophilized and diluted in saline in the range comprised between 10 mg/l to 25 mg/l.

17. A pharmaceutical composition for the use in the treatment of colorectal cancer refractory to previous anticancer therapies and/or for the use in the treatment of patients with peripheral neurotoxicity induced by previous anticancer treatments and/or other neurological distress conditions related to dysregulation of miR-221, including demyelinating disease, comprising the inhibitor LNA-i-miR-221 produced as a lyophilized and diluted in saline at 17.5 mg/l, administered intravenously.

18. The pharmaceutical composition according to claim 14 and possible variants comprising an inhibitor of miR-221, including LNA-i-miR-221, in the presence of excipients of different nature or conveyed with vectors of different nature, for the use in the treatment of patients with human neoplasms refractory to anticancer treatments, including colorectal cancers, alone or in combination with other drugs administered intravenously or by other method of administration.

19. The pharmaceutical composition according to claim 14 comprising an inhibitor of miR-221, including LNA-i-miR-221, in the presence of excipients of different nature or conveyed with vectors of different nature for the use in the treatment of patients with peripheral neurotoxicity induced by previous anticancer treatments and/or for the treatment of other neuropathic disorders attributable to dysregulation of miR-221, including demyelinating disease, alone or in combination with other drugs administered intravenously or by other method of administration.

20. The pharmaceutical composition according to claim 14 for the treatment of patients with refractory solid neoplasms, administered intravenously for 4 consecutive days.


SEQUENCE LISTING (1 sequence)

SEQ ID NO: 1 - DNA, 13 bp, Homo sapiens
  source 1..13: mol_type = genomic DNA
cagacaatgtagc